(19) United States
(12) Patent Application Publication
(10) Pub. No.: US 2007/0129294 A1
(43) Pub. Date: Jun. 7, 2007

(54) Compound and method of treatment

(76) Inventors: Heather Cavanagh, New South Wales (AU); Leath Sheales, New South Wales (AU)

Correspondence Address: STINSON MORRISON HECKER LLP;ATTN: PATENT GROUP, 1201 WALNUT STREET, SUITE 2800, KANSAS CITY, MO, 64106-2150, US

(21) Appl. No.: 10/576,042

(22) PCT Filed: Oct. 18, 2004
(86) PCT No.: PCT/AU04/01430

§ 371 Date: Jan. 24, 2007

(30) Foreign Application Priority Data
Oct. 17, 2003 (AU) 20003905718

(51) Int. Cl. A61K 38/16; A61K 38/10; A61K 38/08; C07K 14/47; C07K 7/08

(52) U.S. Cl. 514/12; 530/324; 530/326; 530/328; 514/14; 514/16; 536/23.5; 435/254.2; 435/320.1

(57) ABSTRACT

The invention relates to a peptide suitable for treatment of tinea and to processes for producing peptides and compositions, for treatment of tinea.

### FIELD OF THE INVENTION

The invention relates to a peptide suitable for treatment of tinea and to processes for producing peptides and compositions, for treatment of tinea.

### BACKGROUND OF THE INVENTION

The many forms of tinea, including tinea capitis, tinea pedis, tinea ungunium, tinea versicolor, tinia cruris, tinea corporis and tinea barbae, are typically caused by fungi of genera Trichopyton, Microsporum and Epidermophyton. Particularly important amongst these fungi are T. tonsurans, M. canis, M. auclounii and T. mentagrophytes, as these species cause tinea capitis, a disease that is one of the most common fungal infections among children and that has reached epidemic proportions throughout the world.

Oral administration of griseofulvin in accordance with a long term dosage schedule is the standard therapy for tinea. As long term dosage schedules tend to be associated with a significant risk of non compliance, a limitation of griseofulvin therapy is that therapy may not be achieved in circumstances of non compliance. Further, in these circumstances, a tolerance or resistance to griseofulvin may develop.

For these and other reasons, other medicaments for treatment of tinea have come to the fore including terbinafine, itraconazole, ketoconazole and fluconazole. A limitation of these medicaments and also with griseofulvin, is that they tend to mediate or otherwise be associated with drug interactions.

There is a need for improved treatment of tinea and in particular, for improved treatment of tinea capitis.

### DESCRIPTION OF THE INVENTION

The invention seeks to at least minimise one of the above limitations and/or to provide an improvement in the treatment of tinea.

In one aspect, the invention provides a peptide including the sequence: Ala-Ile-Lys-Leu-Val-Gln-Ser-Pro.

In another aspect, the invention provides a peptide including the sequence: Ala-Ile-Lys-Leu-Val-Gln-Ser-Pro-Asn-Gly-Asn-Phe-Ala-Ala-Ser.

In another aspect, the invention provides a peptide including the sequence: Ala-Ile-Lys-Leu-Val-Gln-Ser-Pro-Asn-Gly-Asn-Phe-Ala-Ala-Ser-Phe-Val-Leu-Asp-Gly-Thr-Lys-Trp-Ile-Phe-Lys-Ser-Lys-Tyr-Tyr.

As described herein, the inventors have found that peptides including the sequence Ala-Ile-Lys-Leu-Val-Gln-Ser-Pro have a certain functional activity that is characterised by a capacity to inhibit growth of M. canis, M. gypseum, T. tonsurans, T. rubrum and T. mentagrophytes. This is believed to be the first demonstration of peptides of this class that have antifungal activity against these fungal pathogens.

Typically the peptides consist of about 17 amino acid residues although they may be longer.

The peptide may have a molecular weight of between 750 and about 1700 daltons, or between 1450 and about 1700 daltons.

The inventors recognise that a peptide that includes a sequence that, but for one or more amino acid residues, is essentially the same as the sequence Ala-Ile-Lys-Leu-Val-Gln-Ser-Pro, would be expected to have a capacity to inhibit growth of these fungal pathogens. These peptides could be made according to the processes described further herein. The capacity of these peptides to inhibit growth of fungi that cause or are otherwise associated with tinea, such as M. canis, M. gypseum, T. tonsurans, T. rubrum and T. mentagrophytes, could be determined by the assays described further herein In view of the above, it will be understood that the invention includes peptides that have an amino acid sequence that is homologous to the sequence Ala-Ile-Lys-Leu-Val-Gln-Ser-Pro or an amino acid sequence that is homologous to the sequence Ala-Ile-Lys-Leu-Val-Gln-Ser-Pro-Asn-Gly-Asn-Phe-Ala-Ala-Ser or an amino acid sequence that is homologous to the sequence Ala-Ile-Lys-Leu-Val-Gln-Ser-Pro-Asn-Gly-Asn-Phe-Ala-Ala-Ser-Phe-Val-Leu-Asp-Gly-Thr-Lys-Trp-Ile-Phe-Lys-Ser-Lys-Tyr-Tyr. These peptides are referred to as “variants”. Further to amino acid sequence homology with either Ala-Ile-Lys-Leu-Val-Gln-Ser-Pro, Ala-Ile-Lys-Leu-Val-Gln-Ser-Pro-Asn-Gly-Asn-Phe-Ala-Ala-Ser or Ala-Ile-Lys-Leu-Val-Gln-Ser-Pro-Asn-Gly-Asn-Phe-Ala-Ala-Ser-Phe-Val-Leu-Asp-Gly-Thr-Lys-Trp-Ile-Phe-Lys-Ser-Lys-Tyr-Tyr, the variants are characterised in terms of a capacity to inhibit growth of fungi that cause or are otherwise associated with tinea, such as M. canis, M. gypseun, T. tonsurans, T. rubrum and T. mentagrophytes, as determined by the assays described herein.

“Homology” with respect to amino acid sequence is defined as the percentage of amino acid residues in a candidate sequence that are identical with the amino acid residues in the sequence Ala-Ile-Lys-Leu-Val-Gln-Ser-Pro, or in the sequence Ala-Ile-Lys-Leu-Val-Gln-Ser-Pro-Asn-Gly-Asn-Phe-Ala-Ala-Ser, or in the sequence Ala-Ile-Lys-Leu-Val-Gln-Ser-Pro-Asn-Gly-Asn-Phe-Ala-Ala-Ser-Phe-Val-Leu-Asp-Gly-Thr-Lys-Trp-Ile-Phe-Lys-Ser-Lys-Tyr-Tyr, after aligning the sequences and introducing gaps if necessary to achieve the maximum identity. No N- or C-terminal extension or deletion in the candidate sequence shall be construed as reducing homology.

Typically a variant is a peptide that has for example, at least about 75% amino acid homology with the sequence Ala-Ile-Lys-Leu-Val-Gln-Ser-Pro or with the sequence Ala-Ile-Lys-Leu-Val-Gln-Ser-Pro-Asn-Gly-Asn-Phe-Ala-Ala-Ser or with the sequence Ala-Ile-Lys-Leu-Val-Gln-Ser-Pro-Asn-Gly-Asn-Phe-Ala-Ala-Ser-Phe-Val-Leu-Asp-Gly-Thr-Lys-Trp-Ile-Phe-Lys-Ser-Lys-Tyr-Tyr. The variant may have at least 80%, more typically, greater than 85% sequence homology, for example, 90% amino acid homology, with the sequence of Ala-Ile-Lys-Leu-Val-Gln-Ser-Pro, Ala-Ile-Lys-Leu-Val-Gln-Ser-Pro-Asn-Gly-Asn-Phe-Ala-Ala-Ser or Ala-Ile-Lys-Leu-Val-Gln-Ser-Pro-Asn-Gly-Asn-Phe-Ala-Ala-Ser-Phe-Val-Leu-Asp-Gly-Thr-Lys-Trp-Ile-Phe-Lys-Ser-Lys-Tyr-Tyr. However, a variant may exhibit less than 50% sequence homology with the sequence of Ala-Ile-Lys-Leu-Val-Gln-Ser-Pro or Ala-Ile-Lys-Leu-Val-Gln-Ser-Pro-Asp-Gly-Asp-Phe-Ala-Ala-Ser and still retain the characteristics of a variant as described herein.

As described herein, peptides of the invention, including variants, may be prepared by chemical synthesis methodologies or by recombinant DNA technology. For example, peptides of the invention can be prepared from monomers using a chemical synthesis methodology based on the sequential addition of amino acid residues, for example as described in Merrifield, J. Am. Chem. Soc., 85: 2149 (1963). These monomers may be naturally occurring residues, or non naturally occurring residues, examples of which are described below. Alternatively, the peptides of the invention, and in particular, a variant, can be prepared by enzymatically or chemically treating a peptide including the sequence Ala-Ile-Lys-Leu-Val-Gln-Ser-Pro. Where the peptides are to be synthesised by recombinant DNA technology, they may be prepared by random or predetermined mutation (eg site directed PCR mutagenesis) of a nucleic acid molecule that encodes an amino acid sequence Ala-Ile-Lys-Leu-Val-Gln-Ser-Pro, or a sequence that has homology with Ala-Ile-Lys-Leu-Val-Gln-Ser-Pro, and expression of the sequence in a host cell to obtain the peptide. This is a particularly useful process for preparing variants. An alternative process is de novo chemical synthesis of a nucleic acid molecule that encodes Ala-Ile-Lys-Leu-Val-Gln-Ser-Pro or a sequence that is homologous to Ala-Ile-Lys-Leu-Val-Gln-Ser-Pro and expression of the sequence in the host cell to obtain the peptide.

The peptides of the invention that are variants of the sequence Ala-Ile-Lys-Leu-Val-Gln-Ser-Pro or Ala-Ile-Lys-Leu-Val-Gln-Ser-Pro-Asn-Gly-Asn-Phe-Ala-Ala-Ser or Ala-Ile-Lys-Leu-Val-Gln-Ser-Pro-Asn-Gly-Asn-Phe-Ala-Ala-Ser-Phe-Val-Leu-Asp-Gly-Thr-Lys-Trp-Ile-Phe-Lys-Ser-Lys-Tyr-Tyr, typically differ in terms of one or more conservative amino acid substitutions in these sequences. Examples of conservative substitutions are shown in Table 1 below.

TABLE 1

| | Exemplary | Preferred Conserva |
|---|---|---|
| Original Residue | Conservative Substitution | Substitution |
| Ala | Val, Leu, Ile | Val |
| Asn | Gln Lys His Phe | Gln |
| Gln | Asn | Asn |
| Gly | Pro | Pro |
| Ile | Leu, Val, Met Ala, | Leu |
| | Phe | |
| Leu | Ile, Val, Met, Ala, Phe | Ile |
| Lys | Arg, Gln, Asn | Arg |
| Phe | Lue, Val, Ile, Ala | Leu |
| Pro | Gly | Gly |
| Ser | Thr | Thr |
| Val | Ile, Leu, Met, Phe, Ala | Leu |
| Asp | Glu | Glu |
| Thr | Ser | Ser |
| Trp | Tyr | Tyr |
| Tyr | Trp Phe Thr Ser | Phe |

Examples of peptides that are variants include peptides selected from the group consisting of:

Aaa-Ile-Lys-Leu-Val-Gln-Ser-Pro wherein Aaa is Ala or Leu or Val;

Ala-Bbb-Lys-Leu-Val-Gln-Ser-Pro wherein Bbb is Leu, Ile, Pro or Val;

Ala-Ile-Ccc-Leu-Val-Gln-Ser-Pro wherein Ccc is Lys, Pro, Asn, Gln or His;

Ala-Ile-Lys-Ddd-Val-Gln-Ser-Pro wherein Ddd is Leu, Ile or Val;

Ala-Ile-Lys-Leu-Eee-Gln-Ser-Pro wherein Eee is Leu, Ile or Val;

Ala-Ile-Lys-Leu-Val-Fff-Ser-Pro wherein Fff is Gln, Asn, His or Lys; and

Ala-Ile-Lys-Leu-Val-Gln-Ggg-Pro wherein Ggg is Ser or Thr.

As noted above, the peptides of the invention may include non naturally occurring amino acid residues. Commonly encountered amino acids which are not encoded by the genetic code, include:

2-amino adipic acid (Aad) for Glu and Asp;

2-aminopimielic acid (Apm) for Glu and Asp;

2-aminobutyric (Abu) acid for Met, Leu, and other aliphatic amino acids;

2-aminoheptanoic acid (Ahe) for Met, Leu and other aliphatic amino acids;

2-aminoisobutyric acid (Aib) for Gly;

cyclohexylalanine (Cha) for Val, and Leu and Ile;

homoarginine (Har) for Arg and Lys;

2,3-diaminopropionic acid (Dpr) for Lys, Arg and His;

N-ethylglycine (EtGly) for Gly, Pro, and Ala;

N-ethylasparigine (EtAsn) for Asn, and Gln;

Hydroxyllysine (Hyl) for Lys;

allohydroxyllysine (AHyl) for Lys;

3-(and 4) hydroxyproline (3Hyp, 4Hyp) for Pro, Ser, and Thr;

alloisoleucine (Alle) for Ile, Leu, and Val;

ρ-amidinophenylalanine for Ala;

N-methylglycine (MeGly, sarcosine) for Gly, Pro, Ala.

N-methylisoleucine (MeIle) for Ile;

Norvaline (Nva) for Met and other aliphatic amino acids;

Norleucine (Nle) for Met and other aliphatic amino acids;

Ornithine (Orn) for Lys, Arg and His;

Citrulline (Cit) and methionine sulfoxide (MSO) for Thr, Asn and Gln;

N-methylphenylalanine (MePhe), trimethylphenylalanine, halo (F, Cl, Br and I) phenylalanine, triflourylphenylalanine, for Phe.

A useful method for identification of a residue of the sequences Ala-Ile-Lys-Leu-Val-Gln-Ser-Pro, Ala-Ile-Lys-Leu-Val-Gln-Ser-Pro-Asn-Gly-Asn-Phe-Ala-Ala-Ser and Ala-Ile-Lys-Leu-Val-Gln-Ser-Pro-Asn-Gly-Asn-Phe-Ala-Ala-Ser-Phe-Val-Leu-Asp-Gly-Thr-Lys-Trp-Ile-Phe-Lys-Ser-Lys-Tyr-Tyr for amino acid substitution to generate a variant is called alanine scanning mutagenesis as described by Cunningham and Wells (1989) Science, 244:1081-1085. Here a residue or group of target residues are identified (eg charged residues such as Asn, Gln and Lys) and replaced by a neutral or negatively charged amino acid to affect the interaction of the amino acids with the surrounding environment. Those domains demonstrating functional sensitivity to the substitution then are refined by introducing further or other variations at or for the sites of substitution. Thus while the site for introducing an amino acid sequence variation is predetermined the nature of the mutation per se need not be predetermined. For example, to optimize the performance of a mutation at a given site, Ala scanning or random mutagenesis may be conducted at the target codon or region and the expressed peptide screened for the optimal combination of desired activity.

Phage display of protein or peptide libraries offers another methodology for the selection of peptide with improved or altered affinity, specificity, or stability (Smith, G, P, (1991) Curr Opin Biotechnol (2:668-673). High affinity proteins, displayed in a monovalent fashion as fusions with the M13 gene III coat protein (Clackson, T, (1994) et al, Trends Biotechnol 12:173-183), can be identified by cloning and sequencing the corresponding DNA packaged in the phagemid particles after a number of rounds of binding selection.

The peptides of the invention may be prepared as the free acid or base or converted to salts of various inorganic and organic acids and bases. Such salts are within the scope of this invention. Examples of such salts include ammonium, metal salts like sodium, potassium, calcium and magnesium; salts with organic bases like dicyclohexylamine, N-methyl-D-glucamine and the like; and salts with amino acids like arginine or lysine. Salts with inorganic and organic acids may be likewise prepared, for example, using hydrochloric, hydroboronic, sulfuric, phosphoric, trifluoroacetic, methanesulfonic, malic, maleic, fumaric and the like. Non-toxic and physiologically compatible salts are particularly useful, although other less desirable salts may have use in the processes of isolation and purification.

In one embodiment, the peptide includes at least one carbohydrate molecule and/or at least one lipid molecule.

In one embodiment the peptide includes a N-terminal fatty acid moiety, for example a C14 to C17 fatty acid of n, iso or antieso form.

In one embodiment, the peptide includes at least one akyl group.

The peptides of the invention may include a further peptide, for example for controlling degradation of the peptide, or for arranging the peptide on a solid phase or for binding with an antibody or receptor to purify, isolate or detect the peptide. Such peptides are otherwise known in the art as fusion proteins.

Fusion proteins can be made by the chemical synthesis methods describe below, or they can be made by recombinant DNA techniques, for example, wherein a nucleic acid molecule encoding the peptide having the sequence Ala-Ile-Lys-Leu-Val-Gln-Ser-Pro, or the sequence Ala-Ile-Lys-Leu-Val-Gln-Ser-Pro-Asn-Gly-Asn-Phe-Ala-Ala-Ser or the sequence Ala-Ile-Lys-Leu-Val-Gln-Ser-Pro-Asn-Gly-Asn-Phe-Ala-Ala-Ser-Phe-Val-Leu-Asp-Gly-Thr-Lys-Trp-Ile-Phe-Lys-Ser-Lys-Tyr-Tyr is arranged in a vector with a gene encoding another protein or a fragment of another protein. Expression of the vector results in the peptide of the invention being produced as a fusion with another protein or peptide.

The further protein or peptide that is fused to the peptide of the invention may be a protein or peptide that can be secreted by a cell, making it possible to isolate and purify the peptide of the invention from the culture medium and eliminating the necessity of destroying the host cells; this necessity arises when the peptide of the invention remains inside the cell. Alternatively, the fusion protein can be expressed inside the cell as a function of the further protein or peptide. It is useful to use fusion proteins that are highly expressed.

The use of fusion proteins, though not essential, can facilitate the expression of heterologous peptides in E. coli as well as the subsequent purification of those gene products. Harris, in Genetic Engineering, Williamson, R., Ed. (Academic Press, London, Vol. 4, 1983), p. 127; Ljungquist et al., Eur. J. Biochem., 186: 557-561 (1989) and Ljungquist et al., Eur. J. Biochem., 186: 563-569 (1989). Protein A fusions are often used because the binding of protein A, or more specifically the Z domain of protein A, to IgG provides an “affinity handle” for the purification of the fused protein. It has also been shown that many heterologous proteins are degraded when expressed directly in E. coli, but are stable when expressed as fusion proteins. Marston, Biochem J., 240: 1 (1986).

Fusion proteins can be cleaved using chemicals, such as cyanogen bromide, which cleaves at a methionine, or hydroxylamine, which cleaves between an Asn and Gly residue. Using standard recombinant DNA methodology, the nucleotide base pairs encoding these amino acids maybe inserted just prior to the 5′ end of the gene encoding the desired peptide.

Alternatively, one can employ proteolytic cleavage of fusion protein, see for example Carter in Protein Purification: From Molecular mechanisms to Large-Scale Processes, Ladisch et al., eds. (American Chemical Society Symposium Series No. 427, 1990), Ch 13, pages 181-193.

Proteases such as Factor Xa, thrombin, and subtilisin or its mutants, and a number of others have been successfully used to cleave fusion proteins. Typically, a peptide linker that is amenable to cleavage by the protease used is inserted between the further proteins (e.g., the Z domain of protein A) and the peptide of the invention. Using recombinant DNA methodology, the nucleotide base pairs encoding the linker are inserted between the genes or gene fragments coding for the other proteins. Proteolytic cleavage of the partially purified fusion protein containing the correct linker can then be carried out on either the native fusion protein, or the reduced or denatured fusion protein.

The peptide of the invention may not be properly folded when expressed as a fusion protein. Also, the specific peptide linker containing the cleavage site may or may not be accessible to the protease. These factors determine whether the fusion protein must be denatured and refolded, and if so, whether these procedures are employed before or after cleavage.

When denaturing and refolding are needed, typically the peptide is treated with a chaotrope, such as guanidine HCl, and is then treated with a redox buffer, containing, for example, reduced and oxidized dithiothereitol or glutathione at the appropriate ratios, pH, and temperature, such that the peptide is refolded to its native structure.

Other fusion proteins of the invention include those wherein the peptide of the invention is fused to a protein having a long half-life such as immunoglobulin constant region or other immunoglobulin regions, albumin, or ferritin.

The peptides of the invention may be stabilized by polymerization. This may be accomplished by cross linking the peptides with polyfunctional cross linking agents, either directly or indirectly, through multi-functional polymers. For example, two substantially identical polypeptides may be cross linked at their C- or N-termini using a bifunctional cross linking agent. The agent may be used to cross link the terminal amino and/or carboxyl groups. While both terminal carboxyl groups or both terminal amino groups may be cross linked to one another, other cross linking agents permit the alpha amino of one peptide to be cross linked to the terminal carboxyl group of the other peptide.

To facilitate use of other reagents for cross linking, the peptides of the invention may be substituted at their C-termini with cysteine. Under conditions well known in the art a disulfide bond can be formed between the terminal cysteines, thereby cross linking peptide chains. For example, disulfide bridges are conveniently formed by metal-catalyzed oxidation of the free cysteines or by nucleophilic substitution of a suitably modified cysteine residue.

Selection of the cross linking agent will depend upon the identities of the reactive side chains of the amino acids present in the peptides. For example, disulfide cross linking would not be preferred if cysteine was present in the peptide at additional sides other than the C-terminus.

A further approach for cross linking peptides is the use of methylene bridges.

Suitable cross linking sites on the peptides, aside from the N-terminal amino and C-terminal carboxyl groups, include epsilon amino groups found on lysine residues, as well as amino, imino, carboxyl, sulfhydryl and hydroxyl groups located on the side chains of internal residues of the peptides or residues introduced into flanking sequences. Cross linking through externally added cross linking agents is suitably achieved, e.g., using any of a number of reagents familiar to those skilled in the art, for example, via carbodiinide treatment of the peptide. Other examples of suitable multi-functional (ordinarily bifunctional) cross linking agents are found in the literature.

The peptides of this invention also may be conformationally stabilized by cyclization. The peptides may be cyclized by covalently bonding the N- and the C-terminal domains of one peptide to the corresponding domain of another peptide of this invention so as to form cyclo-oligomers containing two or more iterated peptide sequences. Further, cyclized peptides (whether cyclo-oligomers or cyclo-monomers) may be cross linked to form 1-3 cyclic structures having from 2 to 6 peptides comprised therein. The peptides typically are not covalently bonded through α-amino and main chain carboxyl groups (head to tail), but rather are crosslinked through the side chains of residues located in the N- and C-terminal domains. The linking sites thus generally will be between the side chains of the residues.

Many suitable methods per se are known for preparing mono- or poly-cyclized peptides as contemplated herein. Lys/Asp cyclization has been accomplished using Nα-Boc-amino acids on solid-phase support with Fmoc/9-fluorenylmethyl (OFm) side-chain protection for Lys/Asp; the process is completed by piperidine treatment followed by cyclization.

Glu and Lys side chains also have been crosslinked in preparing cyclic or bicyclic peptides: the peptide is synthesized by solid phase chemistry on a p-methylbenzhydrylamine resin. The peptide is cleaved from the resin and deprotected. The cyclic peptide is formed using disphenylphosphrylazide in diluted methylformamide. For an alternative procedure, see Schiller et al., Peptide Protein Res., 25: 171-177 (1985). See also U.S. Pat. No. 4,547,489.

Disulfide cross linked or cyclized peptides may be generated by conventional methods. The method of Pelton et al. (J. Med. Chem., 29: 2370-2375 (1986) is suitable. The same chemistry is useful for synthesis of dimers or cyclo-oliogomers or cyclo-monomers. Also useful are thiomethylene bridges. Lebl and Hruby, Tetrahedron Letters, 25: 2067-2068 (1984). See also Cody et al., J. Med. Chem., 28: 583 (1985).

The desired cyclic or polymeric peptides may be purified by gel filtration followed by reversed-phase high pressure liquid chromatography or other conventional procedures. The peptides may be sterile filtered for formulation into a therapeutic composition described further herein.

The peptides of the invention described above can be made by chemical synthesis or by employing recombinant DNA technology. These methods are known in the art. Chemical synthesis, especially solid phase synthesis, is preferred for short (e.g., less than 50 residues) peptides or those containing unnatural or unusual amino acids such as D-Tyr, Ornithine, amino adipic acid, and the like. Recombinant procedures are preferred for longer peptides. When recombinant procedures are selected, a synthetic gene may be constructed de novo or a natural gene may be mutated by, for example, cassette mutagenesis. These procedures are described further herein. Set forth below are exemplary general procedures for chemical synthesis of peptides of the invention.

Peptides are typically prepared using solid-phase synthesis, such as that generally described by Merrifield, J. Am. Chem. Soc., 85: 2149 (1963), although other equivalent chemical syntheses known in the art are employable. Solid-phase synthesis is initiated from the C-terminus of the peptide by coupling a protected α-amino acid to a suitable resin. Such a starting material can be prepared by attaching a α-amino-protected amino acid by an ester linkage to a chloromethylated resin or a hydroxymethyl resin, or by an amide bond to a BHA resin or MBHA resin. The preparation of the hydroxymethyl resin is described by Bodansky et al., Chem. Ind. (London), 38: 1597-1598 (1966). Chloromethylated resins are commercially available from BioRad Laboratories, Richmond, Calif. And from Lab. Systems, Inc. The preparation of such a resin is described by Stewart et al., “Solid Phase Peptide Synthesis” (Freeman & Co., San Francisco 1969), Chapter 1, pp. 1-6. BHA and MBHA resin supports are commercially available and are generally used only when the desired polypeptide being synthesized has an unsubstituted amide at the C-terminus.

The amino acids are coupled to the peptide chain using techniques well known in the art for the formation of peptide bonds. One method involves converting the amino acid to a derivative that will render the carboxyl group more susceptible to reaction with the free N-terminal amino group of the peptide fragment. For example, the amino acid can be converted to a mixed anhydride by reaction of a protected amino acid with ethychloroformate, phenyl chloroformate, sec-butyl chloroformate, isobutyl chloroformate, pivaloyl chloride or like acid chlorides. Alternatively, the amino acid can be converted to an active ester such as a 2,4,5-trichlorophenyl ester, a pentachlorophenyl ester, a pentafluorophenyl ester, a p-nitrophenyl ester, a N-hydroxysuccinimide ester, or an ester formed from 1-hydroxybenzotriazole.

Another coupling method involves use of a suitable coupling agent such as N,N¹-dicyclohexylcarbodiimide or N,N¹-diisopropylcarbodiimide. Other appropriate coupling agents, apparent in those skilled in the art, are disclosed in E Gross & J Meienhofer, The Peptides: Analysis, Structure, Biology, Vol. I: Major Methods of Peptide Bond Formation (Academic Press, New York, 1979).

It should be recognized that the α-amino group of each amino acid employed in the peptide synthesis must be protected during the coupling reaction to prevent side reactions involving their active α-amino function. It should also be recognized that certain amino acids contain reactive side-chain functional groups (eg sulfhydryl, amino, carboxyl, and hydroxyl) and that such functional groups must also be protected with suitable protecting groups to prevent a chemical reaction from occurring at that site during both the initial and subsequent coupling steps. Suitable protecting groups, known in the art, are described in Gross and Meienhofer, The Peptides: Analysis, Structure, Biology, Vol. 3: “Protection of Functional Groups in Peptide Synthesis” (Academic Press, New York 1981).

In the selection of a particular side-chain protecting group to be used in synthesizing the peptides, the following general rules are followed. An α-amino protecting group must render the α-amino function inert under the conditions employed in the coupling reacting, must be readily removable after the coupling reaction under conditions that will not remove side-chain protecting groups and will not alter the structure of the peptide fragment, and must eliminate the possibility of racemization upon activation immediately prior to coupling. A side-chain protecting group must render the side chain functional group inert under the conditions employed in the coupling reaction, must be stable under the conditions employed in removing the α-amino protecting group, and must be readily removable upon completion of the desired amino acid peptide under reaction conditions that will not alter the structure of the peptide chain.

It will be apparent to those skilled in the art that the protecting groups known to be useful for peptide synthesis will vary in reactivity with the agents employed for their removal. For example, certain protecting groups such as triphenylmethyl and 2-(p-biphenylyl)isopropyloxycarbonyl are very labile and can be cleaved under mild acid conditions. Other protecting groups, such as t-butyloxycarbonyl (BOC), t-amyloxycarbonyl, adamantyloxycarbonyl, and p-methoxybenzyloxycarbonyl are less labile and require moderately strong acid, such as trifluoroacetic, hydrochloric, or boron trifluoride in acetic acid, for their removal. Still other protecting groups, such as benzyloxy-carbonyl (CBZ or Z), halobenzyloxycarbonyl, p-nitrobenzyloxycarbonyl cycloalkyloxycarbonyl, and isopropyloxycarbonyl, are even less labile and require stronger acids, such as hydrogen fluoride, hydrogen bromide, or boron trifluoroacetate in trifluoroacetic acid, for their removal. Among the classes of useful amino acid protecting groups are included:

(1) for an α-amino group, (a) aromatic urethane-type protecting groups, such as fluorenylmethyloxycarbonyl (FMOC) CBZ, and substituted CBZ, such as, eg, p-chlorobenzyloxycarbonyl, p-6-nitrobenzyloxycarbonyl, p-bromobenzyloxycarbonyl, and p-methoxybenzyloxycarbonyl, o-chlorobenzyloxycarbonyl, 2,4-dichlorobenzyloxycarbonyl, 2,6-dichlorobenzyloxycarbonyl, and the like; (b) aliphatic urethane-type protecting groups, such as BOC, t-amyloxycarbonyl, isopropyloxycarbonyl, 2-(p-biphenylyl)-isopropyloxycarbonyl, allyloxycarbonyl and the like; (c) cycloalkyl urethane-type protecting groups, such as cyclopentyloxycarbonyl, adamantyloxycarbonyl, and cyclohexyloxycarbonyl; and (d) allyloxycarbonyl. The preferred α-amino protecting groups are BOX or FMOC.

(2) for the side chain amino group present in Lys, protection may be by any of the groups mentioned above in (1) such as BOC, p-chlorobenzyloxycarbonyl, etc.

(3) for the guanidino group of Arg, protection may be by mitro, tosyl, CBZ, adamantyloxycarbonyl, 2,2,5,7,8-pentamethylchroman-6-sulfonyl or 2,3,6-trimethyl-4-methoxyphenylsulfonyl, or BOC.

(4) for the hydroxyl group of Ser, Thr, or Tyr, protection may be, for example, by C1-C4 alkyl, such as t-butyl; benzyl (BAL); substituted BZL, such as p-methoxybenzyl, p-nitrobenzyl, p-chlorobenzyl, o-chlorobenzyl, and 2,6-dichlorobenzyl.

(5) for the carboxyl group of Asp or Glu, protection may be, for example, by esterification using groups such as BZL, t-butyl, cyclohexyl, cyclopentyl, and the like.

(6) for the imidazole nitrogen of His, the tosyl moiety is suitable employed.

(7) for the phenolic hydroxyl group of Tyr, a protecting group such as tetrahydropyranyl, tert-butyl, trityl, BZL, chlorobenzyl, 4bromobenzyl, or 2,6-dichlorobenzyl is suitably employed. The preferred protecting group is 2,6-dichlorobenzyl.

(8) for the side chain amino group of Asn or Gln, xanthyl (Xan) is preferably employed.

(9) for Met, the amino acid is preferably left unprotected.

(10) for the thio group of Cys, p-methoxybenzyl is typically employed.

The C-terminal amino acid, eg, Lys, is protected at the N-amino position by an appropriately selected protecting group, in the case of Lys, BOC. The BOC-Lys-OH can be first coupled to the benzyhydrylamine or chloromethylated resin according to the procedure set forth in Horiki et al, Chemistry Letters, 165-168 1978) or using isopropylcarbodiimide at about 25° C. for 2 hours with stiring. Following the coupling of the BOC-protected amino acid to the resin support, the α-amino protecting group is removed, as by using trifluoroacetic acid (TFA) in methylene chloride or TFA alone. The deprotection is carried out at a temperature between about 0° C. and room temperature. Other standard cleaving reagents, such as HCl in dioxane, and conditions for removal of specific α-amino protecting groups are described in the literature.

After removal of the α-amino protecting group, the remaining α-amino and side-chain protected amino acids are coupled stepwise within the desired order. As an alternative to adding each amino acid separately in the synthesis, some maybe coupled to one another prior to addition to the solid-phase synthesizer. The selection of an appropriate coupling reagent is within the skill of the art. Particularly suitable as a coupling reagent is N,N¹-dicyclohexyl carbodiimide or diisopropylcarbodiimide.

Each protected amino acid or amino acid sequence is introduced into the solid-phase reactor in excess, and the coupling is suitably carried out in a medium of dimethylformamide (DMF) or CH₂Cl₂ or mixtures thereof. If incomplete coupling occurs, the coupling procedure is repeated before removal of the N-amino protecting group piror to the coupling of the next amino acid. The success of the coupling reaction at each stage of the synthesis may be monitored. A preferred method of monitoring the synthesis is by the ninhydrin reaction, as described by Kaiser et al., Anal Biochem, 34: 595 (1970). The coupling reactions can be performed automatically using well known methods, for example, a BIOSEARCH 9500™ peptide synthesizer.

Upon completion of the desired peptide sequence, the protected peptide must be cleaved from the resin support, and all protecting groups must be removed. The cleavage reaction and removal of the protecting groups is suitably accomplished simultaneously or stepwise. When the resin support is a chloromethylated polystyrene resin, the bond anchoring the peptide to the resin is an ester linkage formed between the free carboxyl group of the C-terminal residue and one of the many chloromethyl groups present on the resin matrix. It will be appreciated that the anchoring bond can be cleaved by reagents that are known to be capable of breaking an ester linkage and of penetrating the resin matrix.

One especially convenient method is by treatment with liquid anhydrous hydrogen fluoride. This reagent not only will cleave the peptide from the resin but also will remove all protecting groups. Hence, use of this reagent will directly afford the fully deprotected peptide. When the chloromethylated resin is used, hydrogen fluoride treatment results in the formation of the free peptide acids. When the benzhydrylamine resin is used, hydrogen fluoride treatment results directly in the free peptide amines. Reaction with hydrogen fluoride in the presence of anisole and dimethylsulfide at 0° C. for one hour will simultaneously remove the side-chain protecting groups and release the peptide from the resin.

When it is desired to cleave the peptide without removing protecting groups, the protected peptide-resin can undergo methanolysis to yield the protected peptide-resin can undergo methanolysis to yield the protected peptide in which the C-terminal carboxyl group is methylated. The methyl ester is then hydrolysed under mild alkaline conditions to give the free C-terminal carboxyl group. The protecting groups on the peptide chain then are removed by treatment with a strong acid, such as liquid hydrogen fluoride. A particularly useful technique for methanolysis is that of Moore et al, Peptides, Proc Fifth Amer Pept Symp, M Goodman and J Meienhofer, Eds, (John Wiley, N.Y., 1977), p. 518-521, in which the protected peptide-resin is treated with methanol and potassium cyanide in the presence of crown ether.

Another method of cleaving the protected peptide form the resin when the chloromethylated resin is employed is by ammonolysis or by treatment with hydrazine. If desired, the resulting C-terminal amide or hydrazide can be hydrolysed to the free C-terminal carboxyl moiety, and the protecting groups can be removed conventionally.

It will also be recognized that the protecting group present on the N-terminal α-amino group may be removed preferentially either before or after the protected peptide is cleaved from the support.

If in the peptides being created carbon atoms bonded to four non identical substituents are asymmetric, then the compounds may exist as disastereoisomers, enantiomers or mixtures thereof The syntheses described above may employ racemates, enantiomers or disastereoisomers as starting materials or intermediates. Disastereomeric products resulting from such syntheses may be separated by chromatographic or crystallization methods. Likewise, enantiomeric product mixtures may be separated using the same techniques or by other methods known in the art. Each of the asymmetric carbon atoms, when present, may be in one of two configurations (R or S) and both are within the scope of the present invention.

Purification of the peptide is typically achieved using conventional procedures such as preparative HPLC (including reversed phase HPLC) or other known chromatographic techniques such as gel permeation, ion exchange, partition chromatography, affinity chromatography (including monoclonal antibody columns) or counter-current distribution.

As described above, the peptide of the invention may be prepared as salts of various inorganic and organic acids and bases. A number of methods are useful for the preparation of these salts and are known to those skilled in the art. Examples include reaction of the free acid or free base form of the peptide with one or more molar equivalents of the desired acid or base in a solvent or solvent mixture in which the salt is insoluble; or in a solvent like water after which the solvent is removed by evaporation, distillation or freeze drying. Alternatively, the free acid or base form of the produce may be passed over an ion-exchange resin to form the desired salt or one salt form of the product may be convened to another using the same general process.

The starting materials required for use in the chemical synthesis of peptides described above are known in the literature or can be prepared using known methods and known starting materials.

The invention also provides a nucleic acid molecule that encodes a peptide according to the invention. Examples of these nucleic acid molecules include molecules having a nucleotide sequence selected from the group consisting of:

(i)
5′-GCU AUC AAA CUG GUU CAG UCC CCG-3′;
(ii)
5′-GCU AUC AAA CUG GUU CAG UCC CCG AAC GGU AAC UUC
GCU GCU UCC-3′;
and
(iii)
5′-GCU AUC AAA CUG GUU CAG UCC CCG AAC GGU AAC UUC
GCU GCU UCC UUC GUU CUG GAC GGU ACC AAA UGG AUC
UUC AAA UCC AAA UAC UAC-3′.

In view of the well known degeneracy of the genetic code, it will be understood that the nucleic acid molecules of the invention may contain one or more points of nucleotide sequence difference, and in particular, one or more codons that are different to the above described nucleic acid molecules. Thus for example, the nucleic molecule that encodes Ala-Ile-Lys-Leu-Val-Gln-Ser-Pro may include the sequence 5′-GCA AUU AAG CUC GUA CAA UCU CCA-3′, rather than 5-‘GCU AUC AAA CUG GUU CAG UCC CCG-3′.

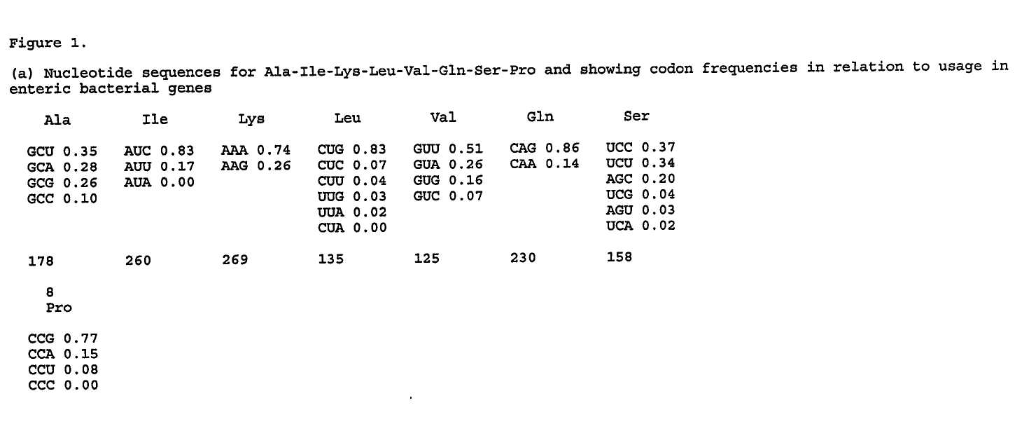

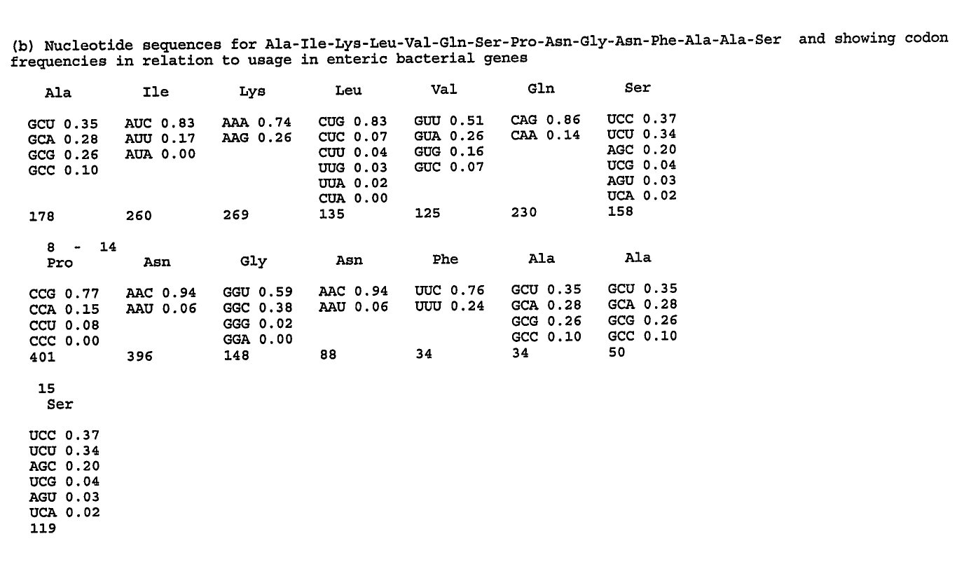

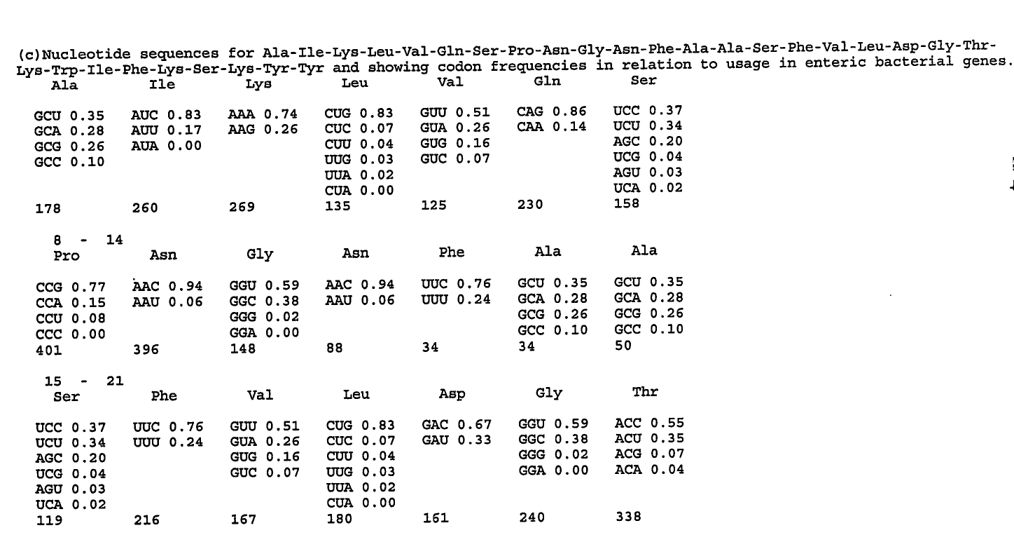

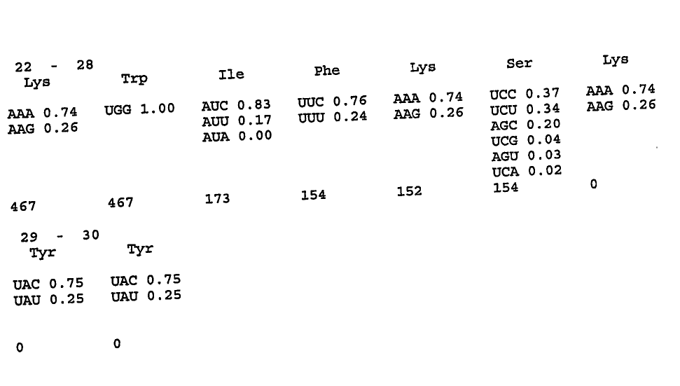

Other exemplary nucleic acid molecules of the invention are described in FIG. 1.

The invention also provides a nucleic acid molecule including a sequence that is complementary to the sequence of a nucleic acid molecule that encodes a peptide according to the invention. An example of such a molecule is 3′-CGA TAG TTT GAC CAA GTC AGG GGC-5′.

A nucleic acid molecule that can hybridise to a molecule having one of the above described nucleotide sequences in high stringency conditions is particularly useful as the complementary strand of this nucleic acid molecule may well encode a peptide of the invention that is a variant. As is well known in the art, hybridisation of nucleic acid molecules may be controlled by the type of buffer used for hybridisation and the temperature of the buffer. “High stringency conditions” are conditions in which the buffer includes about 0.1×SSC, 0.1% SDS and the temperature is about 60° C.

The above described nucleic acid molecules can be obtained from genomic DNA, for example by PCR amplification, from a genomic library, from cDNA derived from mRNA, from a cDNA library, or by synthetically constructing the DNA sequence using synthetically derived nucleotides; (Sambrook et al., Molecular Cloning: A Laboratory Manual (2d ed.), Cold Spring Harbour laboratory, N.Y., 1989).

The nucleic acid molecule of the invention may be a deoxyribonucleotide, a ribonucleotide, a peptide nucleic acid or a combination thereof.

The invention also provides a vector or construct including a nucleic acid molecule of the invention.

The vector or construct is typically obtained by inserting a nucleic acid molecule of the invention into an appropriate plasmid or vector which can be used to transform a cell, for example, a host cell. In general, plasmid vectors containing replication and control sequences which are derived from species compatible with the host cell are used in connection with those hosts. The vector ordinarily carries a replication site, as well as sequences which encode proteins or peptides that are capable of providing phenotypic selection in transformed cells.

For example, E. coli may be transformed using pBR322, a plasmid derived from an E. coli species, see for example Mandel et al., J. Mol. Biol. 53: 154 (1970). Plasmid pBR322 contains genes for ampicillin and tetracycline resistance and thus provides for easy means for selection. Other vectors include different features such as different promoters, which are often important in expression. For example, plasmids pKK223-3, pDR720, and pPL-lambda represent expression vectors with the tac, trp, or P_{L} promoters that are currently available (Pharmacia Biotechnology).

A useful vector is pB0475. This vector contains origins of replication for phage and E. coli that allow it to be shuttled between such hots, thereby facilitating both mutagenesis and expression, see for example, Cunningham et al., Science, 243: 1330-1336 (1989); U.S. Pat. No. 5,580,723. Other useful vectors are pR1T5 and pR1T2T (Pharmacia Biotechnology). These vectors contain appropriate promoters followed by the Z domain of protein A, allowing genes inserted into the vectors to be expressed as fusion proteins.

Other useful vectors can be constructed using standard techniques by combining the relevant traits of the vectors described above. Relevant traits include the promoter, the ribosome binding site, the decorsin or omatin gene or gene fusion (the Z domain of protein A and decorsin or ornatin and its linker), the antibiotic resistance markers, and the appropriate origins of replication.

The invention also provides a cell including a vector or construct as described above. The host cell may be prokaryotic or eukaryotic.

Prokaryotes may be used for cloning and expressing a nucleic acid molecule of the invention to produce the peptide of the invention. For example, E. coli K12 strain 294 (ATCC No. 31446) may be used as well as E. coli B, E. coli X1776 (ATC No. 31537), and E. coli c600 and c600 hfl, E. coli W3110 (F-,gama-,prototrophic/ATCC No. 27325), bacilli such as Bacillus subtillis, and other Enterobacteriaceae such as Salmonella_{—}typhimurium or Serratia marcesans, and various Pseudomonas species. When expressed by prokaryotes the peptide of the invention may contain an N-terminal methionine or a formyl methionine and may not be glycosylated. In the case of fusion proteins, the N-terminal methionine or formyl methionine may reside on the amino terminus of the fusion protein or the signal sequence of the fusion protein.

In addition to prokaryotes, eukaryotic organisms, such as yeast cultures, or cells derived from multicellular organisms may be used. In principle, any such cell culture is workable. However, interest has been greatest in vertebrate cells, and propagation of vertebrate cells in culture (tissue culture) has become reproducible procedure, see for example, Tissue Culture, Academic Press, Kruse and Patterson, editors (173). Examples of such useful host cell lines are VERO and HeLa cells, Chinese Hamster Ovary (CHO) cells lines, W138, 293, BHK, COS-7 and MDCK cell lines.

The invention also provides a process for producing a peptide of the invention. The process includes maintaining a cell containing a nucleic acid molecule as described above, or a vector or construct as described above, in conditions for permitting the cell to produce the peptide.

The process may optionally include the step of recovering and or purifying the protein. Purification of the peptide is typically achieved using conventional procedures such as preparative HPLC (including reversed phase HPLC) or other known chromatographic techniques such as gel permeation, ion exchange, partition chromatography, affinity chromatography (including monoclonal antibody columns) or counter-current distribution.

The invention also provides a composition suitable for treatment of tinea, the composition including the peptide of the invention.

A particularly useful composition is one that can be applied topically to skin. Such a composition includes a peptide of the invention and further includes a solid, semi-solid or liquid, physiologically acceptable vehicle for permitting the peptide to be conveyed to the skin in an appropriate amount. The nature of the vehicle will depend upon the method chosen for topical administration of the composition. The vehicle can itself be inert or it can possess physiological or pharmaceutical benefits of its own.

A vehicle is a substance that can act as a diluent, dispersant or solvent for the peptide of the invention which therefore ensures that it can be applied to and distributed evenly over the desired region of skin at the appropriate concentration. The vehicle is preferably one which can aid penetration of the peptide into the lesion to reach the immediate environment of infection. Compositions according to this invention can include water as a vehicle, and/or at least one acceptable vehicle other than water.

Vehicles other than water that can be used in compositions according to the invention can include solids or liquids such as emollients, solvents, humectants, thickeners and powders. Examples of each of these types of vehicles, which can be used singly or as mixtures of one or more vehicles follows:

emollients such as stearyl alcohol, glyceryl monoricinoleate, glyceryl monostearate, propane-1,2-diol, butane-1,3-diol, mink oil, cetyl alcohol, isopropyl isostearate, stearic acid, isobutyl palmitate, isocetyl sterate, oleyl alcohol, isopropyl laurate, hexyl laurate, decyl oleate, octadecan-2-ol, isocetyl alcohol, eicosanyl alcohol, behenyl alcohol, cetyl palmitate, dimethylpolysiloxane, di-n-butyl sebactate, isopropyl myristate, isopropyl palmitate, isopropyl sterate, butyl stearate, polyethylene glycol, triethylene glycol, lanolin, sesame oil, coconut oil, arachis oil, castor oil, acetylated lanolin alcohols, petroleum, mineral oil, butyl myristate, isostearic acid, palmitic acid, isopropyl linoleate, lauryl lactate, myristyl lactate, decyl oleate, myristyl myristate;

propellants such as trichlorofluoromethane, dichlorodifluormethane, dichlorotetrafluoroethane, monochlorodifluoromethane, trichlorotrifluoroethane, propane, butane, isobutane, dimethyl ether, carbon dioxide, nitrous oxide;

solvents such as ethyl alcohol, methylene chloride, isopropanol, acetone, castor oil, ethylene glycol monoethyl ether, diethylene glycol monobutyl ether, diethyl glycol monoethyl ether, dimethyl sulphoxide, dimethyl formamide, tetrahydrofuran;

humectants such as glycerin, sorbitol, sodium 2-pyrrolidone 5 carboxylate, soluble collagen, dibutyl phthalate, gelatin;

powders such as chalk, talc, fillers earth, kaolin, starch, gums, colloidal silicon dioxide, sodium polyacrylate, tetra alkyl and/or trialykl aryl ammonium smectites, chemically modified magnesium aluminium silicate, organically modified montmorillonite clay, hydrated aluminium silicate, fumed silica, caboxyvinyl polymer, sodium carboxymethyl cellulose, ethylene glycol monstearate.

The composition of the invention can be formulated as a liquid, for example as a lotion, shampoo, conditioner or milk for use in conjunction with an applicator such as a roll ball applicator, or a spray device such as an aerosol can containing propellant, or a container fitted with a pump to dispense the liquid product.

The composition of the invention can also be solid or semi-solid, for example a stick, cream, ointment or gel, for use in conjunction with a suitable application or simply a tube, bottle or lidded jar, or as a liquid impregnated fabric, such as a bandage or tissue wipe.

The invention also provides use of a peptide of the invention for preparing a composition for treatment of tinea.

The amount of peptide of the invention to be used in the composition for topical used can vary widely, but in general, an amount of from 0.0002 to 10 mg/ml is useful.

The following formulation represents a lotion that could be used topically:

TABLE 2

| | % w/v |
|---|---|
| Hydroxyethyl cellulose | 0.4 |
| absolute ethanol | 5 |
| butane-1,3-diol | 38.4 |
| paramethyl benzoate | 0.2 |
| peptide | 0.000005 |
| water | to 100 |

The invention also provides a process for treating an individual for tinea, the process including the step of administering a peptide as described above, to the individual.

The invention also provides a process for controlling the growth of a fungus that is capable of causing tinea, the process including the step of contacting the fungus with a peptide as described above.

### EXAMPLE

Suppression of Fungal Growth Using Peptide of the Invention.

Antifungal activity was assessed by either standard well diffusion assays or standard spore germination assays.

Well Diffusion Assay

Briefly, 100 μL of each test peptide was added to 8 mm wells bored into agar plates prior to inoculation of the agar plate with a plug of actively growing fungus. Controls consisted of agar plates with 100 μL sterile saline added to wells. Sterile saline was consistently utilised for all re-suspensions and dilutions. All plates were incubated at 25° C. until growth of the test fungus (M. canis, M. gypseum, T. tonsurans, T. rubrum and T. mentagrophytes) reached the control wells (time varies according to fungal strain) and the distance from the well to the fungal margin was measured, in mm, for all wells. Results are expressed as percent inhibition relative to control wells.

Peptides were considered as showing a capacity to inhibit M. canis, M. gypseum, T. tonsurans, T. rubrum and T. mentagrophytes provided that the percentage inhibition observed was greater than the control wells. The peptides Ala-Ile-Lys-Leu-Val-Gln-Ser-Pro, Ala-Ile-Lys-Leu-Val-Gln-Ser-Pro-Asn-Gly-Asn-Phe-Ala-Ala-Ser and Ala-Ile-Lys-Leu-Val-Gln-Ser-Pro-Asn-Gly-Asn-Phe-Ala-Ala-Ser-Phe-Val-Leu-Asp-Gly-Thr-Lys-Trp-Ile-Phe-Lys-Ser-Lys-Tyr-Tyr showed greater than 50% and up to 97% inhibition of growth of M. canis, M. gypseum, T. tonsurans, T. rubrum and T. mentagrophytes.

Spore Germination Assay

100 μL of varying concentrations of samples was added to a sterile 96-well microtitre plate and dried at 40° C. in a vacuum oven. 100 μL of spore suspension (1×106 spores/mL) was added to each well of the treated microtitre plates before sealing and incubating in an orbital mixer incubator at 25° C./150 rpm. After 18 hours, spore germination was examined by counting a total of 200 spores and determ ing the number that possessed germ tubes of length equal to that of the spore diameter. The results were reported as percent spores germinated. Control wells had 100 μL of 70% methanol substituted for test substance in the case of testing bacterial extracts. 100 μL of TE buffer was utilised as a control for sepharose fractions and 100 μL sterile saline for purified compound (which was resuspended in sterile saline).

Peptides were considered as showing a capacity to inhibit M. canis, M. gypseum, T. tonsurans, T. rubrum and T. mentagrophytes provided that the percentage inhibition observed was greater than the control wells. The peptides Ala-Ile-Lys-Leu-Val-Gln-Ser-Pro, Ala-Ile-Lys-Leu-Val-Gln-Ser-Pro-Asn-Gly-Asn-Phe-Ala-Ala-Ser and Ala-Ile-Lys-Leu-Val-Gln-Ser-Pro-Asn-Gly-Asn-Phe-Ala-Ala-Ser-Phe-Val-Leu-Asp-Gly-Thr-Lys-Trp-Ile-Phe-Lys-Ser-Lys-Tyr-Tyr showed greater than 50% and up to 97% inhibition of growth of M. canis, M. gypseum, T. tonsurans, T. rubrum and T. mentagrophytes.

1. A peptide including a sequence selected from the group consisting of:
(a)
Ala-Ile-Lys-Leu-Val-Gln-Ser-Pro
(b)
Ala-Ile-Lys-Leu-Val-Gln-Ser-Pro-Asn-Gly-Asn-Phe-
Ala-Ala-Ser
(c)
Ala-Ile-Lys-Leu-Val-Gln-Ser-Pro-Asn-Gly-Asn-Phe-
Ala-Ala-Ser-Phe-Val-Leu-Asp-Gly-Thr-Lys-Trp-Ile-
Phe-Lys-Ser-Lys-Tyr-Tyr.

2. A nucleic acid including a sequence that encodes a peptide according to claim 1.

3. A vector including a nucleic acid according to claim 2.

4. A cell including a vector according to claim 3.

5. A composition for treatment of tinea including a peptide according to claim 1.

6. A process for treating an individual for tinea including the step of administering to an individual a peptide according to claim 1.

7. A process for controlling the growth of a fungus that is capable of causing tinea including the step of contacting a fungus that is capable of causing tinea with a peptide according to claim 1.
